(19) United States
(12) Patent Application Publication
(10) Pub. No.: US 2026/0144822 A1
(43) Pub. Date: May 28, 2026

(54) Treatment of Burn Patients with Hypoxic Blood

(71) Applicant: Hemanext Inc., Lexington, MA (US)

(72) Inventors: Martin CANNON, Chevy Chase, MD (US); Andrew DUNHAM, Tower Lakes, IL (US); Laurel OMERT, Malvern, PA (US)

(73) Assignee: Hemanext Inc., Lexington, MA (US)

(21) Appl. No.: 19/121,673

(22) PCT Filed: Oct. 20, 2023
(86) PCT No.: PCT/US2023/077483

§ 371 Date: Apr. 16, 2025

Related U.S. Application Data
(60) Provisional application No. 63/418,030, filed on Oct. 20, 2022.

(51) Int. Cl. A61K 35/18; A61P 43/00

(52) U.S. Cl. CPC A61K 35/18; A61P 43/00

(57) ABSTRACT

The present disclosure relates to methods for treating burn patients with oxygen reduced blood.

### FIELD OF THE INVENTION

The present disclosure relates to treatment of burn patients with oxygen and carbon dioxide reduced blood.

### BACKGROUND OF THE INVENTION

Major burn trauma has been found to trigger an inflammatory cascade of events which leads to oxidative stress and tissue damage. These injurious responses, which include complement activation, excessive histamine release, decreased blood pressure, and release of reactive oxygen species, may ultimately lead to the development of multiple organ dysfunction. See Wen et al., “Effect of Mitochondrial Antioxidant (Mito-TEMPO) on Burn-Induced Cardiac Dysfunction,” J Am Coll Surg 232:642e655 (2021). Cardiac dysfunction and acute kidney injury, for example, have been well described as sequelae of burn injury and contribute to multi-organ failure. See Jeschke et al., “Long-term persistance of the pathophysiologic response to severe burn injury,” PLoS One; 6:e21245 (2011); Guillory et al., “Cardiovascular dysfunction following burn injury: what we have learned from rat and mouse models,” Int J Mol Sci 17[1](2016); Jeschke et al., “Burn injury,” Nat Rev Dis Primers 6:11 (2020).

Early excision and grafting of large burn wounds are currently performed to attenuate the post-burn hypermetabolic state and remove the biological nidus for infection. These operative procedures can result in considerable bleeding. In one report, intraoperative blood loss was estimated at approximately 9.2% of blood volume for every 1% of total body surface area (TBSA) burn excised. See Steadman et al., “A quantitative assessment of blood loss in burn wound excision and grafting,” Burns: Journal of the International Society for Burn Injuries 18(6):490-491 (1992). Burn surgeons also report progressive development of microvascular bleeding and evidence of coagulopathy requiring plasma and platelets as well as RBC transfusions. See Pidcoke et al., “Acute Blood Loss during Burn and Soft Tissue Excisions: An Observational Study of Blood Product Resuscitation Practices and Focused Review,” J Trauma Acute Care Surg 78(6):S39-S47 (2015). Coagulopathic bleeding post-op can continue for several days, requiring additional transfusions.

Here, we demonstrate for the first time that oxygen and carbon dioxide reduced, leukoreduced blood manufactured using a Hemanext ONE system may decrease the required number of RBC transfusions, increase the time between transfusions for transfusion-dependent patients, reduce the risk and occurrence of transfusion-related iron overload, and reduce adverse events in burn patients. Here, we also demonstrate that in burn patients, hypoxic RBC transfusions can be provided early in resuscitation if there is concomitant trauma, during excision and grafting, and during the post-op period of coagulopathic “oozing”. Hypoxic blood presents the potential to alleviate oxidative stress, improve oxygen delivery, and improve resuscitation efficiency with fewer RBC transfusions. The clinical ramifications that are observed include decreased organ dysfunction in these critically ill patients as well as reduction in thromboembolic events secondary to the improved deformability of the cells.

### SUMMARY

The present specification addresses the need to develop effective transfusion treatments for burn patients.

The present disclosure provides for, and includes, methods for reducing the risk or preventing the occurrence of a transfusion-related adverse event in a burn patient in need thereof comprising administering at least two units of oxygen and carbon dioxide reduced, leukoreduced red blood cells to the burn patient, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the at least two units of oxygen and carbon dioxide reduced, leukoreduced red blood cells are administered to the burn patient in a single transfusion event, and wherein the transfusion-related adverse does not occur for at least 7 days after the administering.

The present disclosure provides for, and includes, methods for reducing the frequency, volume, or frequency and volume of blood transfusions in a burn patient in need thereof following a surgical excision of burn wounds comprising administering at least two units of oxygen and carbon dioxide reduced, leukoreduced red blood cells to the burn patient during the surgical excision of the burn wounds, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the at least two units of oxygen and carbon dioxide reduced, leukoreduced red blood cells are administered during the surgical excision of the burn wounds, wherein the average frequency of subsequent blood transfusions after the administering is reduced by at least 10%, the average volume of subsequent blood transfusions after the administering is reduced by at least 10% and the frequency is reduced by at least 10%, or both the average frequency of subsequent blood transfusions after the administering is reduced by at least 10% and the average volume of subsequent blood transfusions after the administering is reduced by at least 10%.

The present disclosure provides for, and includes, use of donor blood to manufacture oxygen and carbon dioxide reduced, leukoreduced red blood cells having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume for a burn patient in need thereof.

The present disclosure provides for, and includes, use of donor blood to manufacture oxygen and carbon dioxide reduced, leukoreduced red blood cells having an oxygen saturation of (SO₂) of 20% or less and an initial partial pressure of carbon dioxide (pCO₂) of 30 millimeters of mercury (mmHg) or less prior to and during storage for the therapeutic application of increasing the blood transfusion interval for a burn patient in need thereof.

The present disclosure provides for, and includes, use of donor blood to manufacture oxygen and carbon dioxide reduced, leukoreduced red blood cells having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume, increasing the blood transfusion interval, or both in a burn patient in need thereof.

### BRIEF DESCRIPTION OF THE DRAWINGS

The present disclosure is provided with reference to the accompanying drawings, wherein:

FIG. 1 displays the overall study design for a single center, pilot clinical investigation of burn patients, who are transfused with hypoxic red blood cells manufactured with a Hemanext ONE system.

### DETAILED DESCRIPTION

BLOOD PARAMETERS: Methods of the present disclosure provide for, and include, administering oxygen reduced blood to a burn patient in need thereof. In an aspect, the oxygen reduced blood has an initial oxygen saturation (SO₂) of 25% or less and is maintained at an SO₂ of 25% or less for a storage period. In an aspect, the oxygen reduced blood has an initial oxygen saturation (SO₂) of 20% or less and is maintained at an SO₂ of 20% or less for a storage period. In an aspect, the oxygen reduced blood has an initial SO₂ of 15% or less and is maintained at an SO₂ of 15% or less for a storage period. In an aspect, the oxygen reduced blood has an initial SO₂ of 10% or less and is maintained at an SO₂ of 10% or less for a storage period. In an aspect, the oxygen reduced blood has an initial SO₂ of 5% or less and is maintained at an SO₂ of 5% or less for a storage period. In an aspect, the oxygen reduced blood is carbon dioxide reduced. In an aspect, the oxygen reduced blood has an initial partial pressure of carbon dioxide (pCO₂) of 30 millimeters of mercury (mmHg) or less and is maintained at pCO₂ of 30 mmHg or less for a storage period. In an aspect, In an aspect, the oxygen reduced blood has an initial pCO₂ of 20 mmHg or less and is maintained at pCO₂ of 20 mmHg or less for a storage period. In an aspect, In an aspect, the oxygen reduced blood has an initial pCO₂ of 15 mmHg or less and is maintained at pCO₂ of 15 mmHg or less for a storage period. In an aspect, In an aspect, the oxygen reduced blood has an initial pCO₂ of 10 mmHg or less and is maintained at pCO₂ of 10 mmHg or less for a storage period. In an aspect, In an aspect, the oxygen reduced blood has an initial pCO₂ of 5 mmHg or less and is maintained at pCO₂ of 5 mmHg or less for a storage period. In an aspect, oxygen reduced blood is administered to a burn patient in need thereof by transfusion.

Methods of the present disclosure provide for, and include, administering oxygen and carbon dioxide reduced blood to a burn patient in need thereof. In an aspect, the oxygen and carbon dioxide reduced blood has an initial oxygen saturation (SO₂) of 20% or less and an initial partial pressure of carbon dioxide (pCO₂) of 30 millimeters of mercury (mmHg) or less and is maintained at an SO₂ of 20% or less and a pCO₂ of 30 mmHg or less for a storage period. In an aspect, the oxygen and carbon dioxide reduced blood has an initial SO₂ of 20% or less and an initial pCO₂ of 20 mmHg or less and is maintained at an SO₂ of 20% or less and a pCO₂ of 20 mmHg or less for a storage period. In an aspect, the oxygen and carbon dioxide reduced blood has an initial SO₂ of 15% or less and an initial pCO₂ of 20 mmHg or less and is maintained at an SO₂ of 15% or less and a pCO₂ of 20 mmHg or less for a storage period. In an aspect, the oxygen and carbon dioxide reduced blood has an initial SO₂ of 15% or less and an initial pCO₂ of 15 mmHg or less and is maintained at an SO₂ of 15% or less and a pCO₂ of 15 mmHg or less for a storage period. In an aspect, the oxygen and carbon dioxide reduced blood has an initial SO₂ of 10% or less and an initial pCO₂ of 15 mmHg or less and is maintained at an SO₂ of 15% or less and a pCO₂ of 20 mmHg or less for a storage period. In an aspect, the oxygen and carbon dioxide reduced blood has an initial SO₂ of 10% or less and an initial pCO₂ of 10 mmHg or less and is maintained at an SO₂ of 15% or less and a pCO₂ of 10 mmHg or less for a storage period. In an aspect, the oxygen and carbon dioxide reduced blood has an initial SO₂ of 5% or less and an initial pCO₂ of 10 mmHg or less and is maintained at an SO₂ of 5% or less and a pCO₂ of 10 mmHg or less for a storage period. In an aspect, the oxygen and carbon dioxide reduced blood has an initial SO₂ of 5% or less and an initial pCO₂ of 5 mmHg or less and is maintained at an SO₂ of 5% or less and a pCO₂ of 5 mmHg or less for a storage period. In an aspect, oxygen and carbon dioxide reduced blood is administered to a burn patient in need thereof by transfusion.

In an aspect of the present disclosure, oxygen reduced blood is stored for a storage period prior to administration to a burn patient in need thereof. In an aspect, the storage period is less than 2 days, less than 7 days, less than 14 days, less than 21 days, less than 28 days, less than 35 days, less than 42 days, or 42 days. In an aspect, the storage period is 2 days, 7 days, 14 days, 21 days, 28 days, 35 days, or 42 days. In an aspect, the storage period is less than 2 days. In an aspect, the storage period is less than 7 days. In an aspect, the storage period is less than 14 days. In an aspect, the storage period is less than 21 days. In an aspect, the storage period is less than 28 days. In an aspect, the storage period is less than 35 days. In an aspect, the storage period is less than 42 days. In an aspect, the storage period is 42 days.

BLOOD ANTICOAGULANTS AND ADDITIVES: Suitable blood for use in methods according to the present disclosure comprise oxygen reduced blood having an anticoagulant. In an aspect, oxygen reduced blood further comprises an additive solution. Suitable additive solutions according to the present disclosure include AS-1, AS-3 (Nutricel®), AS-5, CPD, SAGM, PAGG-SM, PAGG-GM, MAP, AS-7, ESOL-5, EAS61, OFAS1, OFAS3, and combinations thereof. In an aspect, the additive solution is added at the time of component separation. In an aspect, the additive solution is AS-1. In an aspect, the additive solution is AS-3. In an aspect, the additive solution is SAGM. In an aspect, the additive solution is citrate-phosphate-dextrose (CPD). In an aspect, the additive solution is phosphate-adenosine-guanosine-glucose-saline-mannitol (PAGG-SM). In an aspect, the additive solution is a mixture of citrate-phosphate-dextrose (CPD) and phosphate-adenosine-guanosine-glucose-saline-mannitol (PAGG-SM).

PATIENTS: In an aspect of the present disclosure, a burn patient is in need of a blood transfusion. In an aspect, a burn patient is in need of multiple blood transfusions. In an aspect, a burn patient is in need of periodic blood transfusions. In an aspect, a burn patient is blood transfusion-dependent. As used herein, a blood transfusion-dependent patient is a patient who requires an average of more than two units of blood over a period of at least three weeks. In an aspect, a burn patient is in need of transfusion with oxygen reduced blood. In an aspect, a burn patient has a total body surface burn area of less than 50%. In an aspect, a burn patient has a total body surface burn area of less than 25%. In an aspect, a burn patient has a total body surface burn area of less than 10%. In an aspect, a burn patient has a total body surface burn area of less than 5%. In an aspect, a burn patient has a total body surface burn area of between 5% to 50%. In an aspect, a burn patient has a total body surface burn area of between 10% to 50%. In an aspect, a burn patient is undergoing surgical excision of burn wounds during the administration of oxygen reduced blood to the patient. In an aspect, a burn patient is undergoing surgical grafting of burn wounds during the administration of oxygen reduced blood to the patient. In an aspect, a burn patient is undergoing surgical excision and surgical grafting of burn wounds during the administration of oxygen reduced blood to the patient. In an aspect, a burn patient has undergone surgical excision of burn wounds prior to the administration of oxygen reduced blood to the patient. In an aspect, a burn patient has undergone surgical grafting of burn wounds prior to the administration of oxygen reduced blood to the patient. In an aspect, a burn patient has undergone surgical excision and surgical grafting of burn wounds prior to the administration of oxygen reduced blood to the patient.

In an aspect of the present disclosure, a burn patient has a burn-related complication selected from the group consisting of excessive histamine release, decreased blood pressure, excessive bleeding prior to the administering, and any combination thereof. In an aspect, a burn patient has excessive histamine release. In an aspect, a burn patient has decreased blood pressure. In an aspect, a burn patient has excessive bleeding prior to the administering. In an aspect, a burn patient has one, two, or three burn-related complications. In an aspect, the burn-related complication is improved by at least 10% after being administered oxygen reduced blood. In an aspect, the burn-related complication is improved by at least 20% after being administered oxygen reduced blood. In an aspect, the burn-related complication is improved by at least 30% after being administered oxygen reduced blood. In an aspect, the burn-related complication is improved by at least 40% after being administered oxygen reduced blood. In an aspect, the burn-related complication is improved by at least 50% after being administered oxygen reduced blood. In an aspect, the burn-related complication is improved by at least 60% after being administered oxygen reduced blood. In an aspect, the burn-related complication is improved by at least 70% after being administered oxygen reduced blood. In an aspect, the burn-related complication is improved by at least 80% after being administered oxygen reduced blood. In an aspect, the burn-related complication is improved by at least 90% after being administered oxygen reduced blood. In an aspect, the burn-related complication is improved by at least 95% after being administered oxygen reduced blood. In an aspect, the burn-related complication is improved by between 10% to 25% after being administered oxygen reduced blood. In an aspect, the burn-related complication is improved by between 10% to 50% after being administered oxygen reduced blood. In an aspect, the burn-related complication is improved by between 10% to 90% after being administered oxygen reduced blood. In an aspect, the burn-related complication is improved by between 25% to 50% after being administered oxygen reduced blood. In an aspect, the burn-related complication is improved by between 50% to 90% after being administered oxygen reduced blood. In an aspect, the burn-related complication is improved by at least 10% after being administered oxygen reduced blood compared to a burn patient having been administered the same volume of non-oxygen reduced blood. In an aspect, the burn-related complication is improved by at least 20% after being administered oxygen reduced blood compared to a burn patient having been administered the same volume of non-oxygen reduced blood. In an aspect, the burn-related complication is improved by at least 30% after being administered oxygen reduced blood compared to a burn patient having been administered the same volume of non-oxygen reduced blood. In an aspect, the burn-related complication is improved by at least 40% after being administered oxygen reduced blood compared to a burn patient having been administered the same volume of non-oxygen reduced blood. In an aspect, the burn-related complication is improved by at least 50% after being administered oxygen reduced blood compared to a burn patient having been administered the same volume of non-oxygen reduced blood. In an aspect, the burn-related complication is improved by at least 60% after being administered oxygen reduced blood compared to a burn patient having been administered the same volume of non-oxygen reduced blood. In an aspect, the burn-related complication is improved by at least 70% after being administered oxygen reduced blood compared to a burn patient having been administered the same volume of non-oxygen reduced blood. In an aspect, the burn-related complication is improved by at least 80% after being administered oxygen reduced blood compared to a burn patient having been administered the same volume of non-oxygen reduced blood. In an aspect, the burn-related complication is improved by at least 90% after being administered oxygen reduced blood compared to a burn patient having been administered the same volume of non-oxygen reduced blood. In an aspect, the burn-related complication is improved by between 10% to 25% after being administered oxygen reduced blood compared to a burn patient having been administered the same volume of non-oxygen reduced blood. In an aspect, the burn-related complication is improved by between 10% to 50% after being administered oxygen reduced blood compared to a burn patient having been administered the same volume of non-oxygen reduced blood. In an aspect, the burn-related complication is improved by between 10% to 90% after being administered oxygen reduced blood compared to a burn patient having been administered the same volume of non-oxygen reduced blood. In an aspect, the burn-related complication is improved by between 25% to 50% after being administered oxygen reduced blood compared to a burn patient having been administered the same volume of non-oxygen reduced blood. In an aspect, the burn-related complication is improved by between 50% to 90% after being administered oxygen reduced blood compared to a burn patient having been administered the same volume of non-oxygen reduced blood.

In an aspect of the present disclosure, a burn patient has compartment syndrome, multiple organ dysfunction, or both prior to being administered oxygen reduced blood. In an aspect, a burn patient has compartment syndrome prior to being administered oxygen reduced blood. In an aspect, a burn patient has multiple organ dysfunction prior to being administered oxygen reduced blood. In an aspect, a burn patient has both compartment syndrome and multiple organ dysfunction prior to being administered oxygen reduced blood. In an aspect, the compartment syndrome, multiple organ dysfunction, or both are stabilized after being administered oxygen reduced blood. In an aspect, compartment syndrome in a burn patient is stabilized after being administered oxygen reduced blood. In an aspect, multiple organ dysfunction in a burn patient is stabilized after being administered oxygen reduced blood. In an aspect, both compartment syndrome and multiple organ dysfunction in a burn patient are stabilized after being administered oxygen reduced blood.

BLOOD UNITS/TREATMENT: In an aspect, one, two, or three units of oxygen reduced blood are administered to the burn patient. In an aspect, at least one, at least two, or at least three units of oxygen reduced blood are administered to the burn patient. In an aspect, between one to two, between two to three, or between one to three units of oxygen reduced blood are administered to the burn patient. In an aspect, more than three units of oxygen reduced blood are administered to a burn patient. In an aspect, the oxygen reduced blood is administered to a burn patient as a single transfusion event. In an aspect, the oxygen reduced blood is administered to a burn patient as a double transfusion event. In an aspect, the oxygen reduced blood is administered to a burn patient as a multi-transfusion event.

In an aspect of the present disclosure, a burn patient in need of a blood transfusion is administered oxygen reduced blood periodically over a treatment time period. In an aspect, a treatment time period is for at least 7 days, for at least 14 days, for at least 21 days, or for at least 28 days. In an aspect, the treatment time period is for at least 1 month, for at least 1.5 months, for at least 2 months, for at least 3 months, for at least 4 months, for at least 5 month, for at least 6 months, for at least 7 months, for at least 8 months, for at least 9 months, for at least 10 months, or for at least 11 months. In an aspect, the treatment time period is for at least 1 year, for at least 1.5 years, for at least 2 years, for at least 3 years, for at least 4 years, for at least 5 years, for at least 7.5 years, or for at least 10 years.

In an aspect of the present disclosure, a burn patient in need of a blood transfusion is administered oxygen reduced blood over a transfusion time period. In an aspect, the transfusion time period for a single administration of oxygen reduced blood is at least 30 minutes, at least 1 hour, at least 90 minutes, at least 2 hours, at 3 three hours, at least 4 hours.

In an aspect of the present disclosure, a burn patient in need of a blood transfusion is administered oxygen reduced blood every day, every 2 days, every 3 days, every 4 days, every 5 days, every 6 days, every 7 days, every 8 days, every 9 days, every 10 days, every 12 days, every 14 days, every 16 days, every 18 days, every 20 days, every 22 days, every 24 days, every 26 days, every 28 days, or every 30 days. In an aspect, a burn patient in need of a blood transfusion is administered oxygen reduced blood once a week, twice a week, every other week, every 3rd week, every 4th week, every 5th week, every 6^{th} week, every 7^{th} week, every 8^{th} week, every 9^{th} week, and every 10^{th} week. In an aspect, burn patient in need of a blood transfusion is administered oxygen reduced blood on an irregular basis, or on an as-needed basis.

HEMOGLOBIN: In an aspect of the present disclosure, a burn patient has a hemoglobin level of less than 9 grams per deciliter (g/dL) prior to being administered oxygen reduced blood. In an aspect, a burn patient has a hemoglobin level of less than 8.5 g/dL prior to being administered oxygen reduced blood. In an aspect, a burn patient has a hemoglobin level of less than 8 g/dL prior to being administered oxygen reduced blood. In an aspect, a burn patient has a hemoglobin level of less than 7.5 g/dL prior to being administered oxygen reduced blood. In an aspect, a burn patient has a hemoglobin level of between 8 g/dL to 9 g/dL prior to being administered oxygen reduced blood. In an aspect, a burn patient has a hemoglobin level of about 7.5 g/dL, about 8 g/dL, about 8.5 g/dL, or about 9 g/dL prior to being administered oxygen reduced blood. In an aspect, oxygen reduced blood is oxygen and carbon dioxide reduced blood.

In an aspect of the present disclosure, a burn patient has a hemoglobin level of 9 grams per deciliter (g/dL) or more after being administered oxygen reduced blood. In an aspect, a burn patient has a hemoglobin level of 9.5 g/dL or more after being administered oxygen reduced blood. In an aspect, a burn patient has a hemoglobin level of 10 g/dL or more after being administered to oxygen reduced blood. In an aspect, a burn patient has a hemoglobin level of 11 g/dL or more, 12 g/dL or more, 13 g/dL or more, 14 g/dL or more, 15 g/dL or more, or 16 g/dL or more after being administered oxygen reduced blood. In an aspect, a burn patient has a hemoglobin level of between 9 g/dL to 10 g/dL after being administered oxygen reduced blood.

In an aspect, a burn patient has a hemoglobin level of between 9 g/dL to 12 g/dL after being administered oxygen reduced blood. In an aspect, a burn patient has a hemoglobin level of about 9 g/dL, about 9.5 g/dL, about 10 g/dL, about 11 g/dL, about 12 g/dL, about 13 g/dL, about 14 g/dL, or about 15 g/dL after being administered oxygen reduced blood. In an aspect, a burn patient has a normal hemoglobin level after being administered oxygen reduced blood. In an aspect, oxygen reduced blood is oxygen and carbon dioxide reduced blood.

In an aspect of the present disclosure, the hemoglobin level of a burn patient is increased by at least 0.5 grams per deciliter (g/dL) after being administered oxygen reduced blood. In an aspect, the hemoglobin level of a burn patient is increased by at least 1 g/dL after being administered oxygen reduced blood. In an aspect, the hemoglobin level of a burn patient is increased by at least 1.5 g/dL after being administered oxygen reduced blood. In an aspect, the hemoglobin level of a burn patient is increased by at least 2 g/dL after being administered oxygen reduced blood. In an aspect, the hemoglobin level of a burn patient is increased by at least 3 g/dL, at least 4 g/dL, at least 5 g/dL, or at least 6 g/dL after being administered oxygen reduced blood. In an aspect, the hemoglobin level of a burn patient is increased by between 1 g/dL to 2 g/dL after being administered oxygen reduced blood. In an aspect, the hemoglobin level of a burn patient is increased by between 1 g/dL to 3 g/dL after being administered oxygen reduced blood. In an aspect, the hemoglobin level of a burn patient is increased by at about 1 g/dL after being administered oxygen reduced blood. In an aspect, the hemoglobin level of a burn patient is increased by at about 1.5 g/dL after being administered oxygen reduced blood. In an aspect, the hemoglobin level of a burn patient is increased by at about 2 g/dL after being administered oxygen reduced blood. In an aspect, oxygen reduced blood is oxygen and carbon dioxide reduced blood.

In an aspect of the present disclosure, the hemoglobin level of a burn patient is increased by at least 0.5 grams per deciliter (g/dL) after being administered oxygen reduced blood compared to a burn patient having been administered the same volume of non-oxygen reduced blood. In an aspect, the hemoglobin level of a burn patient is increased by at least 1 g/dL after being administered oxygen reduced blood compared to a burn patient having been administered the same volume of non-oxygen reduced blood. In an aspect, the hemoglobin level of a burn patient is increased by at least 1.5 g/dL after being administered oxygen reduced blood compared to a burn patient having been administered the same volume of non-oxygen reduced blood. In an aspect, the hemoglobin level of a burn patient is increased by at least 2 g/dL after being administered oxygen reduced blood compared to a burn patient having been administered the same volume of non-oxygen reduced blood. In an aspect, the hemoglobin level of a burn patient is increased by at least 3 g/dL, at least 4 g/dL, at least 5 g/dL, or at least 6 g/dL after being administered oxygen reduced blood compared to a burn patient having been administered the same volume of non-oxygen reduced blood. In an aspect, the hemoglobin level of a burn patient is increased by between 1 g/dL to 2 g/dL after being administered oxygen reduced blood compared to a burn patient having been administered the same volume of non-oxygen reduced blood. In an aspect, the hemoglobin level of a burn patient is increased by between 1 g/dL to 3 g/dL after being administered oxygen reduced blood compared to a burn patient having been administered the same volume of non-oxygen reduced blood. In an aspect, the hemoglobin level of a burn patient is increased by at about 1 g/dL after being administered oxygen reduced blood compared to a burn patient having been administered the same volume of non-oxygen reduced blood. In an aspect, the hemoglobin level of a burn patient is increased by at about 1.5 g/dL after being administered oxygen reduced blood compared to a burn patient having been administered the same volume of non-oxygen reduced blood. In an aspect, the hemoglobin level of a burn patient is increased by at about 2 g/dL after being administered oxygen reduced blood compared to a burn patient having been administered the same volume of non-oxygen reduced blood. In an aspect, oxygen reduced blood is oxygen and carbon dioxide reduced blood.

In an aspect, the hemoglobin level of a burn patient is increased by at least 5%, at least 10%, at least 15%, at least 20%, at least 25%, or at least 30% after being administered oxygen reduced blood. In an aspect, the hemoglobin level of a burn patient is increased by at least 1-fold, at least 2-fold, at least 3-fold, at least 4-fold, or at least 5-fold after being administered oxygen reduced blood. In an aspect, the hemoglobin level of a burn patient is increased by at least 5% after being administered oxygen reduced blood compared to a burn patient having been administered the same volume of non-oxygen reduced blood. In an aspect, the hemoglobin level of a burn patient is increased by at least 10% after being administered oxygen reduced blood compared to a burn patient having been administered the same volume of non-oxygen reduced blood. In an aspect, at least 15% after being administered oxygen reduced blood compared to a burn patient having been administered the same volume of non-oxygen reduced blood. In an aspect, the hemoglobin level of a burn patient is increased by at least 20% after being administered oxygen reduced blood compared to a burn patient having been administered the same volume of non-oxygen reduced blood. In an aspect, the hemoglobin level of a burn patient is increased by at least 25% after being administered oxygen reduced blood compared to a burn patient having been administered the same volume of non-oxygen reduced blood. In an aspect, the hemoglobin level of a burn patient is increased by at least 30% after being administered oxygen reduced blood compared to a burn patient having been administered the same volume of non-oxygen reduced blood. In an aspect, the hemoglobin level of a burn patient is increased by at least 1-fold after being administered oxygen reduced blood compared to a burn patient having been administered the same volume of non-oxygen reduced blood. In an aspect, the hemoglobin level of a burn patient is increased by at least 2-fold after being administered oxygen reduced blood compared to a burn patient having been administered the same volume of non-oxygen reduced blood. In an aspect, the hemoglobin level of a burn patient is increased by at least 3-fold after being administered oxygen reduced blood compared to a burn patient having been administered the same volume of non-oxygen reduced blood. In an aspect, the hemoglobin level of a burn patient is increased by at least 4-fold after being administered oxygen reduced blood compared to a burn patient having been administered the same volume of non-oxygen reduced blood. In an aspect, the hemoglobin level of a burn patient is increased by at least 5-fold after being administered oxygen reduced blood compared to a burn patient having been administered the same volume of non-oxygen reduced blood. In an aspect, oxygen reduced blood is oxygen and carbon dioxide reduced blood.

Methods of the present disclosure provide for, and include, increasing the hemoglobin increment of a burn patient in need thereof by administering at least two units of oxygen reduced blood, as provided herein, to a burn patient. As used herein, a hemoglobin increment is the change in hemoglobin per unit of transfused blood. In an aspect, the hemoglobin increment of a burn patient is increased by at least 0.5 grams per deciliter (g/dL) after being administered oxygen reduced blood. In an aspect, the hemoglobin increment of a burn patient is increased by at least 1 g/dL after being administered oxygen reduced blood. In an aspect, the hemoglobin increment of a burn patient is increased by at least 1.5 g/dL after being administered oxygen reduced blood. In an aspect, the hemoglobin increment of a burn patient is increased by at least 2 g/dL after being administered oxygen reduced blood. In an aspect, the hemoglobin increment of a burn patient is increased by at least 2.5 g/dL after being administered oxygen reduced blood. In an aspect, the hemoglobin increment of a burn patient is increased by between 0.5 to 1 g/dL after being administered oxygen reduced blood. In an aspect, the hemoglobin increment of a burn patient is increased by between 1 to 2 g/dL after being administered oxygen reduced blood. In an aspect, the hemoglobin increment of a burn patient is increased by one-fold after being administered oxygen reduced blood. In an aspect, the hemoglobin increment of a burn patient is increased by two-fold after being administered oxygen reduced blood. In an aspect, the hemoglobin increment of a burn patient is increased by three-fold after being administered oxygen reduced blood. In an aspect, the hemoglobin increment of a burn patient is increased by four-fold after being administered oxygen reduced blood. In an aspect, the hemoglobin increment of a burn patient is increased by five-fold after being administered oxygen reduced blood. In an aspect, iron overload is reduced in a burn patient after being administered oxygen reduced blood.

REDUCING OR PREVENTING TRANSFUSION-RELATED ADVERSE EVENTS: Methods of the present disclosure provide for, and include, preventing the occurrence of a transfusion-related adverse event in a burn patient in need thereof comprising administering at least two units of oxygen reduced blood to a burn patient. Methods of the present disclosure provide for, and include, reducing the occurrence of a transfusion-related adverse event in a burn patient in need thereof comprising administering at least two units of oxygen reduced blood to a burn patient. Methods of the present disclosure provide for, and include, preventing or reducing the occurrence of a transfusion-related adverse event in a burn patient in need thereof comprising administering at least two units of oxygen reduced blood to a burn patient. As used herein, an adverse event is an unfavorable event resulting from a blood transfusion. In an aspect of the present disclosure, administering oxygen reduced blood to a burn patient prevents a transfusion-related adverse event for at least 7 days, at least 14 days, at least 21 days, at least 28 days, at least 35 days, or at least 48 days after the administration. In an aspect, administering oxygen reduced blood to a burn patient reduces a transfusion-related adverse event for at least 7 days, at least 14 days, at least 21 days, at least 28 days, at least 35 days, or at least 48 days after the administration. In an aspect, administering oxygen reduced blood to a burn patient prevents or reduces a transfusion-related adverse event for at least 7 days, at least 14 days, at least 21 days, at least 28 days, at least 35 days, or at least 48 days after the administration. In an aspect, no transfusion-related adverse event occurs for at least 7 days after administering oxygen reduced blood to a burn patient. In an aspect, no transfusion-related adverse event occurs for at least 14 days after administering oxygen reduced blood to a burn patient. In an aspect, no transfusion-related adverse event occurs for at least 21 days after administering oxygen reduced blood to a burn patient. In an aspect, no transfusion-related adverse event occurs for at least 28 days after administering oxygen reduced blood to a burn patient. In an aspect, no transfusion-related adverse event occurs for at least 35 days after administering oxygen reduced blood to a burn patient. In an aspect, a transfusion-related adverse event is reduced for at least 7 days after administering oxygen reduced blood to a burn patient. In an aspect, a transfusion-related adverse event is reduced for at least 14 days after administering oxygen reduced blood to a burn patient.

In an aspect, a transfusion-related adverse event is reduced for at least 21 days after administering oxygen reduced blood to a burn patient. In an aspect, In an aspect, a transfusion-related adverse event is reduced for at least 28 days after administering oxygen reduced blood to a burn patient. In an aspect, a transfusion-related adverse event is reduced for at least 35 days after administering oxygen reduced blood to a burn patient.

In an aspect of the present disclosure, a transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), haematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof. In an aspect, the transfusion-related adverse event is iron overload. In an aspect, the transfusion-related adverse event is hemorrhagic shock. In an aspect, the transfusion-related adverse event is alloimmunization. In an aspect, the transfusion-related adverse event is an allergic reaction. In an aspect, the transfusion-related adverse event is a transfusion-transmitted infection (TTI). In an aspect, the transfusion-related adverse event is transfusion-associated graft versus host disease. In an aspect, the transfusion-related adverse event is transfusion-related acute lung injury (TRALI). In an aspect, the transfusion-related adverse event is post-transfusion purpura. In an aspect, the transfusion-related adverse event is transfusion-associated circulatory overload (TACO). In an aspect, the transfusion-related adverse event is transfusion-associated dyspnea (TAD). In an aspect, the transfusion-related adverse event is febrile non-hemolytic transfusion reaction (FNHTR). In an aspect, the transfusion-related adverse event is hypotensive transfusion reaction. In an aspect, the transfusion-related adverse event is delayed hemolytic transfusion reaction (DHTR). In an aspect, the transfusion-related adverse event is delayed serologic transfusion reaction (DSTR). In an aspect, the transfusion-related adverse event is acute hemolytic transfusion reaction (AHTR). In an aspect, the transfusion-related adverse event is a major cardiac event (MCE). In an aspect, the transfusion-related adverse event is haematoma. In an aspect, the transfusion-related adverse event is arterial puncture. In an aspect, the transfusion-related adverse event is delayed bleeding. In an aspect, the transfusion-related adverse event is localized infection or inflammation. In an aspect, the transfusion-related adverse event is brachial artery pseudoaneurysm. In an aspect, the transfusion-related adverse event is deep vein thrombosis (DVT). In an aspect, the transfusion-related adverse event is vasovagal reactions. In an aspect, the transfusion-related adverse event is nerve injury or irritation. In an aspect, the transfusion-related adverse event is compartment syndrome. In an aspect, the transfusion-related adverse event is arteriovenous fistula. In an aspect, a transfusion-related adverse event is a combination of 1, 2, 3, 4, 5, 6, 7, 8, 9, 10, 11, or 12 transfusion-related adverse events.

In an aspect, a transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death. In an aspect, the transfusion-related serious adverse event is a life-threatening illness. In an aspect, the transfusion-related serious adverse event is a life-threatening injury. In an aspect, the transfusion-related serious adverse event is permanent impairment of a body structure. In an aspect, the transfusion-related serious adverse event is permanent impairment of a body function. In an aspect, the transfusion-related serious adverse event is hospitalization. In an aspect, the transfusion-related serious adverse event is prolongation of hospitalization. In an aspect, the transfusion-related serious adverse event is a need for medical or surgical intervention to prevent a life-threatening illness or injury. In an aspect, the transfusion-related serious adverse event is contraction of a chronic disease. In an aspect, the transfusion-related serious adverse event is death. In an aspect, a transfusion-related adverse event is a combination of 1, 2, 3, 4, 5, 6, or 7 serious adverse events.

INCREASING BLOOD TRANSFUSION INTERVAL: Methods of the present disclosure provide for, and include, increasing the blood transfusion interval for a burn patient in need thereof by administering at least two units of oxygen reduced blood, as provided herein, to a burn patient. As used herein, an increased blood transfusion interval means an increase in the period of time between subsequent blood transfusions (i.e., resulting from less frequent blood transfusions administered over time). As used herein, an average interval between blood transfusions refers to the average (mean) time between blood transfusions for a series of two or more blood transfusions. In an aspect, the average interval between subsequent blood transfusions administered to a burn patient is increased by at least 5% compared to the average interval between blood transfusions administered to a burn patient previously transfused with an identical volume of non-oxygen reduced blood. In an aspect, the average interval between subsequent blood transfusions administered to a burn patient is increased by at least 10% compared to the average interval between blood transfusions administered to a burn patient previously transfused with an identical volume of non-oxygen reduced blood. In an aspect, the average interval between subsequent blood transfusions administered to a burn patient is increased by at least 15% compared to the average interval between blood transfusions administered to a burn patient previously transfused with an identical volume of non-oxygen reduced blood. In an aspect, the average interval between subsequent blood transfusions administered to a burn patient is increased by at least 20% compared to the average interval between blood transfusions administered to a burn patient previously transfused with an identical volume of non-oxygen reduced blood. In an aspect, the average interval between subsequent blood transfusions administered to a burn patient is increased by at least 25% compared to the average interval between blood transfusions administered to a burn patient previously transfused with an identical volume of non-oxygen reduced blood. In an aspect, the average interval between subsequent blood transfusions administered to a burn patient is increased by at least 30% compared to the average interval between blood transfusions administered to a burn patient previously transfused with an identical volume of non-oxygen reduced blood. In an aspect, the average interval between subsequent blood transfusions administered to a burn patient is increased by at least 35% compared to the average interval between blood transfusions administered to a burn patient previously transfused with an identical volume of non-oxygen reduced blood. In an aspect, the average interval between subsequent blood transfusions administered to a burn patient is increased by at least 40% compared to the average interval between blood transfusions administered to a burn patient previously transfused with an identical volume of non-oxygen reduced blood. In an aspect, the average interval between subsequent blood transfusions administered to a burn patient is increased by at least 45% compared to the average interval between blood transfusions administered to a burn patient previously transfused with an identical volume of non-oxygen reduced blood. In an aspect, the average interval between subsequent blood transfusions administered to a burn patient is increased by at least 50% compared to the average interval between blood transfusions administered to a burn patient previously transfused with an identical volume of non-oxygen reduced blood. In an aspect, the average interval between subsequent blood transfusions administered to a burn patient is increased by between 10% to 20% compared to the average interval between blood transfusions administered to a burn patient previously transfused with an identical volume of non-oxygen reduced blood. In an aspect, the average interval between subsequent blood transfusions administered to a burn patient is increased by between 10% to 30% compared to the average interval between blood transfusions administered to a burn patient previously transfused with an identical volume of non-oxygen reduced blood. In an aspect, the average interval between subsequent blood transfusions administered to a burn patient is increased by between 10% to 40% compared to the average interval between blood transfusions administered to a burn patient previously transfused with an identical volume of non-oxygen reduced blood. In an aspect, the average interval between subsequent blood transfusions administered to a burn patient is increased by between 10% to 50% compared to the average interval between blood transfusions administered to a burn patient previously transfused with an identical volume of non-oxygen reduced blood. In an aspect, the average interval between subsequent blood transfusions administered to a burn patient is increased by between 20% to 30% compared to the average interval between blood transfusions administered to a burn patient previously transfused with an identical volume of non-oxygen reduced blood. In an aspect, the average interval between subsequent blood transfusions administered to a burn patient is increased by between 20% to 40% compared to the average interval between blood transfusions administered to a burn patient previously transfused with an identical volume of non-oxygen reduced blood. In an aspect, the average interval between subsequent blood transfusions administered to a burn patient is increased by between 20% to 50% compared to the average interval between blood transfusions administered to a burn patient previously transfused with an identical volume of non-oxygen reduced blood. In an aspect, the average interval between subsequent blood transfusions administered to a burn patient is increased by between 30% to 40% compared to the average interval between blood transfusions administered to a burn patient previously transfused with an identical volume of non-oxygen reduced blood. In an aspect, the average interval between subsequent blood transfusions administered to a burn patient is increased by between 30% to 50% compared to the average interval between blood transfusions administered to a burn patient previously transfused with an identical volume of non-oxygen reduced blood. In an aspect, the average interval between subsequent blood transfusions administered to a burn patient is increased by between 5% to 50% compared to the average interval between blood transfusions administered to a burn patient previously transfused with an identical volume of non-oxygen reduced blood. In an aspect, the average interval between subsequent blood transfusions administered to a burn patient is increased at least one-fold compared to the average interval between blood transfusions administered to a burn patient previously transfused with an identical volume of non-oxygen reduced blood. In an aspect, the average interval between subsequent blood transfusions administered to a burn patient is increased at least two-fold compared to the average interval between blood transfusions administered to a burn patient previously transfused with an identical volume of non-oxygen reduced blood. In an aspect, the average interval between subsequent blood transfusions administered to a burn patient is increased at least three-fold compared to the average interval between blood transfusions administered to a burn patient previously transfused with an identical volume of non-oxygen reduced blood. In an aspect, the average interval between subsequent blood transfusions administered to a burn patient is increased at least four-fold compared to the average interval between blood transfusions administered to a burn patient previously transfused with an identical volume of non-oxygen reduced blood. In an aspect, the average interval between subsequent blood transfusions administered to a burn patient is increased at least five-fold compared to the average interval between blood transfusions administered to a burn patient previously transfused with an identical volume of non-oxygen reduced blood. In an aspect, the average interval between subsequent blood transfusions administered to a burn patient is increased by about one week compared to the average interval between blood transfusions administered to a burn patient previously transfused with an identical volume of non-oxygen reduced blood. In an aspect, the average interval between subsequent blood transfusions administered to a burn patient is increased by about two weeks compared to the average interval between blood transfusions administered to a burn patient previously transfused with an identical volume of non-oxygen reduced blood. In an aspect, the average interval between subsequent blood transfusions administered to a burn patient is increased by about three weeks compared to the average interval between blood transfusions administered to a burn patient previously transfused with an identical volume of non-oxygen reduced blood. In an aspect, the average interval between subsequent blood transfusions administered to a burn patient is increased by about four weeks compared to the average interval between blood transfusions administered to a burn patient previously transfused with an identical volume of non-oxygen reduced blood. In an aspect, the average interval between subsequent blood transfusions administered to a burn patient is increased by between one day and one week compared to the average interval between blood transfusions administered to a burn patient previously transfused with an identical volume of non-oxygen reduced blood. In an aspect, the average interval between subsequent blood transfusions administered to a burn patient is increased by between one week and two weeks compared to the average interval between blood transfusions administered to a burn patient previously transfused with an identical volume of non-oxygen reduced blood. In an aspect, the average interval between subsequent blood transfusions administered to a burn patient is increased by between one week and four weeks compared to the average interval between blood transfusions administered to a burn patient previously transfused with an identical volume of non-oxygen reduced blood. In an aspect, the average interval between subsequent blood transfusions administered to a burn patient is increased by between two weeks and four weeks compared to the average interval between blood transfusions administered to a burn patient previously transfused with an identical volume of non-oxygen reduced blood.

In an aspect of the present disclosure, the average interval between blood transfusions administered to a burn patient is increased by at least 5% after administering at least two units of oxygen reduced blood, as provided herein, to a burn patient. In an aspect, the average interval between blood transfusions administered to a burn patient is increased by at least 10% after administering at least two units of oxygen reduced blood, as provided herein, to a burn patient. In an aspect, the average interval between blood transfusions administered to a burn patient is increased by at least 15% after administering at least two units of oxygen reduced blood, as provided herein, to a burn patient. In an aspect, the average interval between blood transfusions administered to a burn patient is increased by at least 20% after administering at least two units of oxygen reduced blood, as provided herein, to a burn patient. In an aspect, the average interval between blood transfusions administered to a burn patient is increased by at least 25% after administering at least two units of oxygen reduced blood, as provided herein, to a burn patient. In an aspect, the average interval between blood transfusions administered to a burn patient is increased by at least 25% after administering at least two units of oxygen reduced blood, as provided herein, to a burn patient. In an aspect, the average interval between blood transfusions administered to a burn patient is increased by at least 30% after administering at least two units of oxygen reduced blood, as provided herein, to a burn patient. In an aspect, the average interval between blood transfusions administered to a burn patient is increased by at least 35% after administering at least two units of oxygen reduced blood, as provided herein, to a burn patient. In an aspect, the average interval between blood transfusions administered to a burn patient is increased by at least 40% after administering at least two units of oxygen reduced blood, as provided herein, to a burn patient. In an aspect, the average interval between blood transfusions administered to a burn patient is increased by at least 45% after administering at least two units of oxygen reduced blood, as provided herein, to a burn patient. In an aspect, the average interval between blood transfusions administered to a burn patient is increased by at least 50% after administering at least two units of oxygen reduced blood, as provided herein, to a burn patient. In an aspect, the average interval between blood transfusions administered to a burn patient is increased at least one-fold after administering at least two units of oxygen reduced blood, as provided herein, to a burn patient. In an aspect, the average interval between blood transfusions administered to a burn patient is increased at least two-fold after administering at least two units of oxygen reduced blood, as provided herein, to a burn patient. In an aspect, the average interval between blood transfusions administered to a burn patient is increased at least three-fold after administering at least two units of oxygen reduced blood, as provided herein, to a burn patient. In an aspect, the average interval between blood transfusions administered to a burn patient is increased at least four-fold after administering at least two units of oxygen reduced blood, as provided herein, to a burn patient. In an aspect, the average interval between blood transfusions administered to a burn patient is increased at least five-fold after administering at least two units of oxygen reduced blood, as provided herein, to a burn patient. In an aspect, the average interval between blood transfusions administered to a burn patient is increased by about one day after administering at least two units of oxygen reduced blood, as provided herein, to a burn patient. In an aspect, the average interval between blood transfusions administered to a burn patient is increased by about two days after administering at least two units of oxygen reduced blood, as provided herein, to a burn patient. In an aspect, the average interval between blood transfusions administered to a burn patient is increased by about three days after administering at least two units of oxygen reduced blood, as provided herein, to a burn patient. In an aspect, the average interval between blood transfusions administered to a burn patient is increased by about four days after administering at least two units of oxygen reduced blood, as provided herein, to a burn patient. In an aspect, the average interval between blood transfusions administered to a burn patient is increased by about five days after administering at least two units of oxygen reduced blood, as provided herein, to a burn patient. In an aspect, the average interval between blood transfusions administered to a burn patient is increased by about six days after administering at least two units of oxygen reduced blood, as provided herein, to a burn patient. In an aspect, the average interval between blood transfusions administered to a burn patient is increased by about one week after administering at least two units of oxygen reduced blood, as provided herein, to a burn patient. In an aspect, the average interval between blood transfusions administered to a burn patient is increased by about two weeks after administering at least two units of oxygen reduced blood, as provided herein, to a burn patient. In an aspect, the average interval between blood transfusions administered to a burn patient is increased by about three weeks after administering at least two units of oxygen reduced blood, as provided herein, to a burn patient. In an aspect, the average interval between blood transfusions administered to a burn patient is increased by about four weeks after administering at least two units of oxygen reduced blood, as provided herein, to a burn patient. In an aspect, the average interval between blood transfusions administered to a burn patient is increased by between one day and one week after administering at least two units of oxygen reduced blood, as provided herein, to a burn patient. In an aspect, the average interval between blood transfusions administered to a burn patient is increased by between one week and two weeks after administering at least two units of oxygen reduced blood, as provided herein, to a burn patient. In an aspect, the average interval between blood transfusions administered to a burn patient is increased by between one day to one week after administering at least two units of oxygen reduced blood, as provided herein, to a burn patient. In an aspect, the average interval between blood transfusions administered to a burn patient is increased by between one week to two weeks after administering at least two units of oxygen reduced blood, as provided herein, to a burn patient. In an aspect, the average interval between blood transfusions administered to a burn patient is increased by between one week and four weeks after administering at least two units of oxygen reduced blood, as provided herein, to a burn patient. In an aspect, the average interval between blood transfusions administered to a burn patient is increased by between two weeks to four weeks after administering at least two units of oxygen reduced blood, as provided herein, to a burn patient.

REDUCING BLOOD TRANSFUSION VOLUME: Methods of the present disclosure provide for, and include, reducing the blood transfusion volume for a burn patient in need thereof by administering at least two units of oxygen reduced blood, as provided herein, to a burn patient. As used herein, a reduced blood transfusion volume is a reduction in the volume of blood administered after an initial administration. As used herein, an average interval between blood transfusions refers to the average (mean) volume of a series of two or more blood transfusions. In an aspect, the average blood transfusion volume of subsequent blood transfusions administered to a burn patient is reduced by at least 5% compared to the average blood transfusion volume administered to a burn patient previously transfused with an identical volume of non-oxygen reduced blood. In an aspect, the average blood transfusion volume of subsequent blood transfusions administered to a burn patient is reduced by at least 10% compared to the average blood transfusion volume administered to a burn patient previously transfused with an identical volume of non-oxygen reduced blood. In an aspect, the average blood transfusion volume of subsequent blood transfusions administered to a burn patient is reduced by at least 15% compared to the average blood transfusion volume administered to a burn patient previously transfused with an identical volume of non-oxygen reduced blood. In an aspect, the average blood transfusion volume of subsequent blood transfusions administered to a burn patient is reduced by at least 20% compared to the average blood transfusion volume administered to a burn patient previously transfused with an identical volume of non-oxygen reduced blood. In an aspect, the average blood transfusion volume of subsequent blood transfusions administered to a burn patient is reduced by at least 25% compared to the average blood transfusion volume administered to a burn patient previously transfused with an identical volume of non-oxygen reduced blood. In an aspect, the average blood transfusion volume of subsequent blood transfusions administered to a burn patient is reduced by at least 30% compared to the average blood transfusion volume administered to a burn patient previously transfused with an identical volume of non-oxygen reduced blood. In an aspect, the average blood transfusion volume of subsequent blood transfusions administered to a burn patient is reduced by at least 35% compared to the average blood transfusion volume administered to a burn patient previously transfused with an identical volume of non-oxygen reduced blood. In an aspect, the average blood transfusion volume of subsequent blood transfusions administered to a burn patient is reduced by at least 40% compared to the average blood transfusion volume administered to a burn patient previously transfused with an identical volume of non-oxygen reduced blood. In an aspect, the average blood transfusion volume of subsequent blood transfusions administered to a burn patient is reduced by at least 45% compared to the average blood transfusion volume administered to a burn patient previously transfused with an identical volume of non-oxygen reduced blood. In an aspect, the average blood transfusion volume of subsequent blood transfusions administered to a burn patient is reduced by at least 50% compared to the average blood transfusion volume administered to a burn patient previously transfused with an identical volume of non-oxygen reduced blood. In an aspect, the average blood transfusion volume of subsequent blood transfusions administered to a burn patient is reduced by between 10% to 20% compared to the average blood transfusion volume administered to a burn patient previously transfused with an identical volume of non-oxygen reduced blood. In an aspect, the average blood transfusion volume of subsequent blood transfusions administered to a burn patient is reduced by between 10% to 30% compared to the average blood transfusion volume administered to a burn patient previously transfused with an identical volume of non-oxygen reduced blood. In an aspect, the average blood transfusion volume of subsequent blood transfusions administered to a burn patient is reduced by between 10% to 40% compared to the average blood transfusion volume administered to a burn patient previously transfused with an identical volume of non-oxygen reduced blood. In an aspect, the average blood transfusion volume of subsequent blood transfusions administered to a burn patient is reduced by between 10% to 50% compared to the average blood transfusion volume administered to a burn patient previously transfused with an identical volume of non-oxygen reduced blood. In an aspect, the average blood transfusion volume of subsequent blood transfusions administered to a burn patient is reduced by between 20% to 30% compared to the average blood transfusion volume administered to a burn patient previously transfused with an identical volume of non-oxygen reduced blood. In an aspect, the average blood transfusion volume of subsequent blood transfusions administered to a burn patient is reduced by between 20% to 40% compared to the average blood transfusion volume administered to a burn patient previously transfused with an identical volume of non-oxygen reduced blood. In an aspect, the average blood transfusion volume of subsequent blood transfusions administered to a burn patient is reduced by between 20% to 50% compared to the average blood transfusion volume administered to a burn patient previously transfused with an identical volume of non-oxygen reduced blood. In an aspect, the average blood transfusion volume of subsequent blood transfusions administered to a burn patient is reduced by between 30% to 40% compared to the average blood transfusion volume administered to a burn patient previously transfused with an identical volume of non-oxygen reduced blood. In an aspect, the average blood transfusion volume of subsequent blood transfusions administered to a burn patient is reduced by between 30% to 50% compared to the average blood transfusion volume administered to a burn patient previously transfused with an identical volume of non-oxygen reduced blood. In an aspect, the average blood transfusion volume of subsequent blood transfusions administered to a burn patient is reduced by between 5% to 50% compared to the average blood transfusion volume administered to a burn patient previously transfused with an identical volume of non-oxygen reduced blood. In an aspect, the average blood transfusion volume of subsequent blood transfusions administered to a burn patient is reduced by at least one-fold compared to the average blood transfusion volume administered to a burn patient previously transfused with an identical volume of non-oxygen reduced blood. In an aspect, the average blood transfusion volume of subsequent blood transfusions administered to a burn patient is reduced by at least two-fold compared to the average blood transfusion volume administered to a burn patient previously transfused with an identical volume of non-oxygen reduced blood. In an aspect, the average blood transfusion volume of subsequent blood transfusions administered to a burn patient is reduced by at least three-fold compared to the average blood transfusion volume administered to a burn patient previously transfused with an identical volume of non-oxygen reduced blood. In an aspect, the average blood transfusion volume of subsequent blood transfusions administered to a burn patient is reduced by at least four-fold compared to the average blood transfusion volume administered to a burn patient previously transfused with an identical volume of non-oxygen reduced blood. In an aspect, the average blood transfusion volume of subsequent blood transfusions administered to a burn patient is reduced by at least five-fold compared to the average blood transfusion volume administered to a burn patient previously transfused with an identical volume of non-oxygen reduced blood.

In an aspect, the average transfusion volume of subsequent blood transfusions administered to a burn patient is 5% less than the initial volume of oxygen reduced blood administered to the patient. In an aspect, the average transfusion volume of subsequent blood transfusions administered to a burn patient is 10% less than the initial volume of oxygen reduced blood administered to the patient. In an aspect, the average transfusion volume of subsequent blood transfusions administered to a burn patient is 20% less than the initial volume of oxygen reduced blood administered to the patient. In an aspect, the average transfusion volume of subsequent blood transfusions administered to a burn patient is 30% less than the initial volume of oxygen reduced blood administered to the patient. In an aspect, the average transfusion volume of subsequent blood transfusions administered to a burn patient is 40% less than the initial volume of oxygen reduced blood administered to the patient. In an aspect, the average transfusion volume of subsequent blood transfusions administered to a burn patient is 50% less than the initial volume of oxygen reduced blood administered to the patient. In an aspect, the average transfusion volume of subsequent blood transfusions administered to a burn patient is between 10% to 20% less than the initial volume of oxygen reduced blood administered to the patient. In an aspect, the average transfusion volume of subsequent blood transfusions administered to a burn patient is between 10% to 30% less than the initial volume of oxygen reduced blood administered to the patient. In an aspect, the average transfusion volume of subsequent blood transfusions administered to a burn patient is between 10% to 40% less than the initial volume of oxygen reduced blood administered to the patient. In an aspect, the average transfusion volume of subsequent blood transfusions administered to a burn patient is between 10% to 50% less than the initial volume of oxygen reduced blood administered to the patient. In an aspect, the average transfusion volume of subsequent blood transfusions administered to a burn patient is between 20% to 30% less than the initial volume of oxygen reduced blood administered to the patient. In an aspect, the average transfusion volume of subsequent blood transfusions administered to a burn patient is between 20% to 40% less than the initial volume of oxygen reduced blood administered to the patient. In an aspect, the average transfusion volume of subsequent blood transfusions administered to a burn patient is between 20% to 50% less than the initial volume of oxygen reduced blood administered to the patient. In an aspect, the average transfusion volume of subsequent blood transfusions administered to a burn patient is between 30% to 40% less than the initial volume of oxygen reduced blood administered to the patient. In an aspect, the average transfusion volume of subsequent blood transfusions administered to a burn patient is between 30% to 50% less than the initial volume of oxygen reduced blood administered to the patient. In an aspect, the average transfusion volume of subsequent blood transfusions administered to a burn patient is between 5% to 50% less than the initial volume of oxygen reduced blood administered to the patient. In an aspect, the average transfusion volume of subsequent blood transfusions administered to a burn patient is at least one-fold less than the initial volume of oxygen reduced blood administered to the patient. In an aspect, the average transfusion volume of subsequent blood transfusions administered to a burn patient is at least two-fold less than the initial volume of oxygen reduced blood administered to the patient. In an aspect, the average transfusion volume of subsequent blood transfusions administered to a burn patient is at least three-fold less than the initial volume of oxygen reduced blood administered to the patient. In an aspect, the average transfusion volume of subsequent blood transfusions administered to a burn patient is at least four-fold less than the initial volume of oxygen reduced blood administered to the patient. In an aspect, the average transfusion volume of subsequent blood transfusions administered to a burn patient is at least five-fold less than the initial volume of oxygen reduced blood administered to the patient.

In an aspect, the average transfusion volume of subsequent blood transfusions administered to a burn patient having been previously transfused with an oxygen reduced blood is less than 1 liter (L). In an aspect, the average transfusion volume of subsequent blood transfusions administered to a burn patient having been previously transfused with an oxygen reduced blood is less than 900 milliliters (mL). In an aspect, the average transfusion volume of subsequent blood transfusions administered to a burn patient having been previously transfused with an oxygen reduced blood is less than 800 mL. In an aspect, the average transfusion volume of subsequent blood transfusions administered to a burn patient having been previously transfused with an oxygen reduced blood is less than 700 mL. In an aspect, the average transfusion volume of subsequent blood transfusions administered to a burn patient having been previously transfused with an oxygen reduced blood is less than 600 mL. In an aspect, the average transfusion volume of subsequent blood transfusions administered to a burn patient having been previously transfused with an oxygen reduced blood is less than 500 mL. In an aspect, the average transfusion volume of subsequent blood transfusions administered to a burn patient having been previously transfused with an oxygen reduced blood is less than 400 mL. In an aspect, the average transfusion volume of subsequent blood transfusions administered to a burn patient having been previously transfused with an oxygen reduced blood is less than 250 mL. In an aspect, the average transfusion volume of subsequent blood transfusions administered to a burn patient having been previously transfused with an oxygen reduced blood is between 500 mL and 1 L. In an aspect, the average transfusion volume of subsequent blood transfusions administered to a burn patient having been previously transfused with an oxygen reduced blood is 2 units or less. In an aspect, the average transfusion volume of subsequent blood transfusions administered to a burn patient having been previously transfused with an oxygen reduced blood is 2 units. In an aspect, the average transfusion volume of subsequent blood transfusions administered to a burn patient having been previously transfused with an oxygen reduced blood is 1.5 units or less. In an aspect, the average transfusion volume of subsequent blood transfusions administered to a burn patient having been previously transfused with an oxygen reduced blood is 1.5 units. In an aspect, the average transfusion volume of subsequent blood transfusions administered to a burn patient having been previously transfused with an oxygen reduced blood is 1 unit or less. In an aspect, the average transfusion volume of subsequent blood transfusions administered to a burn patient having been previously transfused with an oxygen reduced blood is 1 unit.

IMPROVING OXYGEN DELIVERY: Methods of the present disclosure provide for, and include, improving oxygen delivery to a burn patient in need thereof comprising administering at least two units of oxygen reduced blood, as provided herein, to a burn patient. As used herein, oxygen delivery/oxygenation is measured by blood oxygen level. In an aspect, the blood oxygen level of a burn patient is at least 5% greater than the blood oxygen level of a burn patient transfused with an identical volume of non-oxygen reduced blood. In an aspect, the blood oxygen level of a burn patient is at least 10% greater than the blood oxygen level of a burn patient transfused with an identical volume of non-oxygen reduced blood. In an aspect, the blood oxygen level of a burn patient is at least 15% greater than the blood oxygen level of a burn patient transfused with an identical volume of non-oxygen reduced blood. In an aspect, the blood oxygen level of a burn patient is at least 20% greater than the blood oxygen level of a burn patient transfused with an identical volume of non-oxygen reduced blood. In an aspect, the blood oxygen level of a burn patient is at least 25% greater than the blood oxygen level of a burn patient transfused with an identical volume of non-oxygen reduced blood. In an aspect, the blood oxygen level of a burn patient is at least 30% greater than the blood oxygen level of a burn patient transfused with an identical volume of non-oxygen reduced blood. In an aspect, the blood oxygen level of a burn patient is at least 35% greater than the blood oxygen level of a burn patient transfused with an identical volume of non-oxygen reduced blood. In an aspect, the blood oxygen level of a burn patient is at least 40% greater than the blood oxygen level of a burn patient transfused with an identical volume of non-oxygen reduced blood. In an aspect, the blood oxygen level of a burn patient is at least 45% greater than the blood oxygen level of a burn patient transfused with an identical volume of non-oxygen reduced blood. In an aspect, the blood oxygen level of a burn patient is at least 50% greater than the blood oxygen level of a burn patient transfused with an identical volume of non-oxygen reduced blood. In an aspect, the blood oxygen level of a burn patient is between 10% to 20% greater than the blood oxygen level of a burn patient transfused with an identical volume of non-oxygen reduced blood. In an aspect, the blood oxygen level of a burn patient is between 10% to 30% greater than the blood oxygen level of a burn patient transfused with an identical volume of non-oxygen reduced blood. In an aspect, the blood oxygen level of a burn patient is between 10% to 40% greater than the blood oxygen level of a burn patient transfused with an identical volume of non-oxygen reduced blood. In an aspect, the blood oxygen level of a burn patient is between 10% to 50% greater than the blood oxygen level of a burn patient transfused with an identical volume of non-oxygen reduced blood. In an aspect, the blood oxygen level of a burn patient is between 20% to 30% greater than the blood oxygen level of a burn patient transfused with an identical volume of non-oxygen reduced blood. In an aspect, the blood oxygen level of a burn patient is between 20% to 40% greater than the blood oxygen level of a burn patient transfused with an identical volume of non-oxygen reduced blood. In an aspect, the blood oxygen level of a burn patient is between 20% to 50% greater than the blood oxygen level of a burn patient transfused with an identical volume of non-oxygen reduced blood. In an aspect, the blood oxygen level of a burn patient is between 30% to 40% greater than the blood oxygen level of a burn patient transfused with an identical volume of non-oxygen reduced blood. In an aspect, the blood oxygen level of a burn patient is between 30% to 50% greater than the blood oxygen level of a burn patient transfused with an identical volume of non-oxygen reduced blood. In an aspect, the blood oxygen level of a burn patient is between 5% to 50% greater than the blood oxygen level of a burn patient transfused with an identical volume of non-oxygen reduced blood.

In an aspect, the blood oxygen level of a burn patient is at least 5% higher than the blood oxygen level of the patient before oxygen reduced blood was administered. In an aspect, the blood oxygen level of a burn patient is at least 10% higher than the blood oxygen level of the patient before oxygen reduced blood was administered. In an aspect, the blood oxygen level of a burn patient is at least 20% higher than the blood oxygen level of the patient before oxygen reduced blood was administered. In an aspect, the blood oxygen level of a burn patient is at least 30% higher than the blood oxygen level of the patient before oxygen reduced blood was administered. In an aspect, the blood oxygen level of a burn patient is at least 40% higher than the blood oxygen level of the patient before oxygen reduced blood was administered. In an aspect, the blood oxygen level of a burn patient is at least 50% higher than the blood oxygen level of the patient before oxygen reduced blood was administered. In an aspect, the blood oxygen level of a burn patient is between 10% to 20% higher than the blood oxygen level of the patient before oxygen reduced blood was administered. In an aspect, the blood oxygen level of a burn patient is between 10% to 30% higher than the blood oxygen level of the patient before oxygen reduced blood was administered. In an aspect, the blood oxygen level of a burn patient is between 10% to 40% higher than the blood oxygen level of the patient before oxygen reduced blood was administered. In an aspect, the blood oxygen level of a burn patient is between 10% to 50% higher than the blood oxygen level of the patient before oxygen reduced blood was administered. In an aspect, the blood oxygen level of a burn patient is between 20% to 30% higher than the blood oxygen level of the patient before oxygen reduced blood was administered. In an aspect, the blood oxygen level of a burn patient is between 20% to 40% higher than the blood oxygen level of the patient before oxygen reduced blood was administered. In an aspect, the blood oxygen level of a burn patient is between 20% to 50% higher than the blood oxygen level of the patient before oxygen reduced blood was administered. In an aspect, the blood oxygen level of a burn patient is between 5% to 50% higher than the blood oxygen level of the patient before oxygen reduced blood was administered.

In an aspect, the blood oxygen level of a burn patient is less than 80% prior to being administered oxygen reduced blood. In an aspect, the blood oxygen level of a burn patient is less than 85% prior to being administered oxygen reduced blood. In an aspect, the blood oxygen level of a burn patient is less than 90% prior to being administered oxygen reduced blood. In an aspect, the blood oxygen level of a burn patient is between 80% to 85% prior to being administered oxygen reduced blood. In an aspect, the blood oxygen level of a burn patient is between 80% to 90% prior to being administered oxygen reduced blood. In an aspect, the blood oxygen level of a burn patient is at least 90% after being administered oxygen reduced blood. In an aspect, the blood oxygen level of a burn patient is at least 95% after being administered oxygen reduced blood. In an aspect, the blood oxygen level of a burn patient is between 90% to 100% after being administered oxygen reduced blood. In an aspect, the blood oxygen level of a burn patient is in normal range after administered oxygen reduced blood.

REDUCING OR PREVENTING TRANSFUSION-RELATED SEQUELAE: Methods of the present disclosure provide for, and include, preventing transfusion-related sequelae in a burn patient in need thereof by administering at least two units of oxygen reduced blood, as provided herein, to a burn patient. Methods of the present disclosure provide for, and include, reducing transfusion-related sequelae in a burn patient in need thereof by administering at least two units of oxygen reduced blood, as provided herein, to a burn patient. Methods of the present disclosure provide for, and include, preventing or reducing transfusion-related sequelae in a burn patient in need thereof by administering at least two units of oxygen reduced blood, as provided herein, to a burn patient. As used herein, transfusion-related sequelae are adverse events resulting from a blood transfusion. In an aspect, administering oxygen reduced blood to a burn patient prevents transfusion-related sequelae for at least 7 days, at least 14 days, at least 21 days, at least 28 days, at least 35 days, or at least 48 days after the administration. In an aspect, administering oxygen reduced blood to a burn patient reduces transfusion-related sequelae for at least 7 days, at least 14 days, at least 21 days, at least 28 days, at least 35 days, or at least 48 days after the administration. In an aspect, administering oxygen reduced blood to a burn patient prevents or reduces transfusion-related sequelae for at least 7 days, at least 14 days, at least 21 days, at least 28 days, at least 35 days, or at least 48 days after the administration. In an aspect, no transfusion-related sequelae occur for 7 days after administering oxygen reduced blood to a burn patient. In an aspect, one transfusion-related sequela occurs over a 7 day period after administering oxygen reduced blood to a burn patient. In an aspect, two transfusion-related sequelae occurs over a 7 day period after administering oxygen reduced blood to a burn patient. In an aspect, two or less transfusion-related sequelae occurs over a 7 day period after administering oxygen reduced blood to a burn patient. In an aspect, three transfusion-related sequelae occurs over a 7 day period after administering oxygen reduced blood to a burn patient. In an aspect, three or less transfusion-related sequelae occurs over a 7 day period after administering oxygen reduced blood to a burn patient. In an aspect, no transfusion-related sequelae occur for 14 days after administering oxygen reduced blood to a burn patient. In an aspect, one transfusion-related sequela occurs over a 14 day period after administering oxygen reduced blood to a burn patient. In an aspect, two transfusion-related sequelae occurs over a 14 day period after administering oxygen reduced blood to a burn patient. In an aspect, two or less transfusion-related sequelae occurs over a 14 day period after administering oxygen reduced blood to a burn patient. In an aspect, three transfusion-related sequelae occurs over a 14 day period after administering oxygen reduced blood to a burn patient. In an aspect, three or less transfusion-related sequelae occurs over a 14 day period after administering oxygen reduced blood to a burn patient. In an aspect, no transfusion-related sequelae occur for 21 days after administering oxygen reduced blood to a burn patient. In an aspect, one transfusion-related sequela occurs over a 21 day period after administering oxygen reduced blood to a burn patient. In an aspect, two transfusion-related sequelae occurs over a 21 day period after administering oxygen reduced blood to a burn patient. In an aspect, two or less transfusion-related sequelae occurs over a 21 day period after administering oxygen reduced blood to a burn patient. In an aspect, three transfusion-related sequelae occurs over a 21 day period after administering oxygen reduced blood to a burn patient. In an aspect, three or less transfusion-related sequelae occurs over a 21 day period after administering oxygen reduced blood to a burn patient. In an aspect, no transfusion-related sequelae occur for 28 days after administering oxygen reduced blood to a burn patient. In an aspect, one transfusion-related sequela occurs over a 28 day period after administering oxygen reduced blood to a burn patient. In an aspect, two transfusion-related sequelae occurs over a 28 day period after administering oxygen reduced blood to a burn patient. In an aspect, two or less transfusion-related sequelae occurs over a 28 day period after administering oxygen reduced blood to a burn patient. In an aspect, three transfusion-related sequelae occurs over a 28 day period after administering oxygen reduced blood to a burn patient. In an aspect, three or less transfusion-related sequelae occurs over a 28 day period after administering oxygen reduced blood to a burn patient.

In an aspect of the present disclosure, transfusion-related sequelae are selected from the group consisting of dyspnea, fever, chills, facial flushing, severe pain, shock, rapid pulse, low blood pressure, hypothermia, respiratory distress, nausea, vomiting, itching, and jaundice. In an aspect of the present disclosure, transfusion-related sequelae include dyspnea, fever, chills, facial flushing, severe pain, shock, rapid pulse, low blood pressure, hypothermia, respiratory distress, nausea, vomiting, itching, jaundice, or any combination thereof. In an aspect, the transfusion-related sequelae comprise dyspnea. In an aspect, the transfusion-related sequelae comprises fever. In an aspect, the transfusion-related sequelae comprise dyspnea. In an aspect, the transfusion-related sequelae comprises chills. In an aspect, the transfusion-related sequelae comprise dyspnea. In an aspect, the transfusion-related sequelae comprise facial flushing. In an aspect, the transfusion-related sequelae comprise severe pain. In an aspect, the transfusion-related sequelae comprises facial flushing. In an aspect, the transfusion-related sequelae comprise shock. In an aspect, the transfusion-related sequelae comprises facial flushing. In an aspect, the transfusion-related sequelae comprise rapid pulse. In an aspect, the transfusion-related sequelae comprises facial flushing. In an aspect, the transfusion-related sequelae comprise low blood pressure. In an aspect, the transfusion-related sequelae comprise hypothermia. In an aspect, the transfusion-related sequelae comprise respiratory distress. In an aspect, the transfusion-related sequelae comprise nausea. In an aspect, the transfusion-related sequelae comprises vomiting. In an aspect, the transfusion-related sequelae comprise itching. In an aspect, the transfusion-related sequelae comprise vomiting. In an aspect, the transfusion-related sequelae comprise jaundice. In an aspect, transfusion-related sequelae comprise a combination of 1, 2, 3, 4, 5, 6, 7, 8, 9, 10, 11, 12, 13, or 14 sequela.

IMPROVING QUALITY OF LIFE: Methods of the present disclosure provide for, and include, improving the quality of life of a burn patient in need thereof by administering at least two units of oxygen reduced blood, as provided herein, to a burn patient. In an aspect, improved quality of life is determined by evaluation of a patient's health-related quality of life (HRQOL) assessment following administration of oxygen reduced blood. HRQOL comprises a series of questions designed to assess a patient's perceived physical and mental health. In an aspect, the quality of life of a burn patient is improved after being administered oxygen reduced blood as assessed by the HRQOL data of the patient. In an aspect, the improved quality of life of a burn patient in need thereof is reduced fatigue, reduced appetite loss, reduced insomnia, reduced stress, reduced anxiety, or any combination thereof. In an aspect, the improved quality of life of a burn patient in need thereof is reduced fatigue. In an aspect, the improved quality of life of a burn patient in need thereof is reduced appetite loss. In an aspect, the improved quality of life of a burn patient in need thereof is reduced insomnia In an aspect, the improved quality of life of a burn patient in need thereof is reduced stress. In an aspect, the improved quality of life of a burn patient in need thereof is reduced anxiety

### Definitions

As used herein, the singular forms “a,” “an,” and “the” include plural references unless the context clearly dictates otherwise. For example, the term “a compound” or “at least one compound” can include a plurality of compounds, including mixtures thereof.

As used herein the term “about” refers to ±10%.

As used herein the “Hemanext ONE” system is a blood container set used to process and store CPD/PAGGSM Red Blood Cells, Leukocytes reduced, and O2/CO2 Reduced. The Hemanext ONE system is an assembly of two disposables: the Oxygen Reduction Bag (ORB) and the Hemanext (Anaerobic) Storage Bag (HSB), as outlined in International Publication Nos. WO 2016/145210 A1 and WO 2016/172645 A1 (incorporated herein by reference).

As used herein, the term “adverse event” is any unfavorable event resulting from a blood transfusion.

As used herein, the term “blood” refers to whole blood, leukoreduced red blood cells (RBCs), platelet reduced RBCs, and leukocyte and platelet reduced RBCs. The term “blood” further includes packed red blood cells, platelet reduced packed red blood cells, leukocyte reduced packed red blood cells, and leukocyte and platelet reduced packed red blood cells. The temperature of blood can vary depending on the stage of the collection process, starting at the normal body temperature of 37° C. at the time and point of collection, but decreasing rapidly to about 30° C. as soon as the blood leaves the patient's body and further thereafter to room temperature in about 6 hours when untreated, and ultimately being refrigerated at between about 4° C. and 6° C. Human red blood cells in vivo are in a dynamic state. The red blood cells contain hemoglobin, the iron-containing protein that carries oxygen throughout the body and gives red blood its color. The percentage of blood volume composed of red blood cells is called the hematocrit. As used herein, unless otherwise limited, RBCs also includes packed red blood cells (pRBCs). Packed red blood cells are prepared from whole blood using centrifugation techniques commonly known in the art. As used herein, unless otherwise indicated, the hematocrit of pRBCs is about 70%. As used herein, oxygen reduced stored RBCs can include oxygen and carbon dioxide reduced stored RBCs. As used herein, oxygen reduced (OR) blood can include oxygen and carbon dioxide (OCR) reduced blood.

As used herein, the term “hypoxic blood” refers to oxygen reduced blood or oxygen and carbon dioxide reduced blood having percent SO₂ and partial pressure of CO₂ as provided herein and in paragraph [0017].

As used herein, the terms “comprises,” “comprising,” “includes,” “including,” “having,” and their conjugates mean “including but not limited to.”

As used herein, the term “consisting of” means “including and limited to.”

As used herein, the term “consisting essentially of” means that the composition, method or structure can include additional ingredients, steps and/or parts, but only if the additional ingredients, steps and/or parts do not materially alter the basic and novel characteristics of the claimed composition, method or structure.

As used herein, the terms “higher”, “greater” or “increased” means that the measured values of oxygen reduced blood, when compared to the measured values of otherwise equivalently treated non-oxygen reduced conventionally stored blood, are at least 1 standard deviation greater, with a sample size of at least 2 for each compared measured condition.

As used herein, the terms “injury”, “damage”, and “failure” refer to an organ not functioning properly or not functioning as is expected in a person or animal without disease or injury.

As used herein, the term “less than” refers to a smaller amount and an amount greater than zero.

As used herein, the terms “patient” and “subject” are used interchangeably to mean a human or animal in need of treatment with the methods disclosed herein.

As used herein, the terms “reduce”, “reduced”, “lower”, “decreased” or “less” means that the measured values of oxygen reduced blood when compared to the measured values of otherwise equivalently treated non-oxygen reduced conventionally stored blood, are at least 1 standard deviation lower, with a sample size of at least 2 for each compared measured condition.

Various aspects of this disclosure may be presented as a fold change (e.g., a fold increase or a fold reduction). Within the context of measured values of oxygen reduced blood when compared to the measured values of otherwise equivalently treated non-oxygen reduced conventionally stored blood in the disclosure, the word “fold” should be understood to entail a multiplication in the case of a fold increase, or a division in the case of a fold reduction. For example, “increased by 2 fold” means a multiplication by 2. As another example, “reduced by 2 fold” means a division by 2.

As used herein, a “unit” of blood is about 450-500 mL including anticoagulant. Suitable anticoagulants include CPD, CPDA1, ACD, and ACD-A.

As used herein, the terms “administer”, “administration”, and “administering” refer to the delivery of blood to a patient, e.g., by transfusion.

Throughout this application, various aspects of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. Description of ranges may include terms such as “from” and “between.” For example, description of a range such as “from 1 to 6” should be considered to have specifically disclosed subranges such as “from 1 to 3,” “from 1 to 4,” “from 1 to 5,” “from 2 to 4,” “from 2 to 6,” “from 3 to 6,” etc., as well as individual numbers within that range, for example, 1, 2, 3, 4, 5, and 6. This applies regardless of the breadth of the range. As another example, description of a range such as “between 1 and 6” should be considered to have specifically disclosed subranges such as “between 1 and 3,” “between 1 and 4,” “between 1 and 5,” “between 2 and 4,” “between 2 and 6,” “between 3 and 6,” etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number “and” a second indicated number, and “ranging/ranges from” a first indicated number “to” a second indicated number are used interchangeably herein and are meant to include the first and second indicated numbers and all the fractional and integral numerals there between. Similarly, the phrases “between” a first indicated number “and” a second indicated number, and “between” a first indicated number “to” a second indicated number are also used interchangeably herein and are meant to include the first and second indicated numbers and all the fractional and integral numerals there between.

As used herein, the term “method” refers to manners, means, techniques, and procedures for accomplishing a given task including, but not limited to, administering to a human burn patient oxygen reduced stored blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period.

### Embodiments

Embodiment 1. A method for reducing the risk or preventing the occurrence of a transfusion-related adverse event in a burn patient in need thereof comprising administering at least two units of oxygen and carbon dioxide reduced blood to the burn patient, the oxygen and carbon dioxide reduced blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the at least two units of oxygen and carbon dioxide reduced blood are administered to the burn patient in a single transfusion event, and wherein the transfusion-related adverse does not occur for at least 7 days after the administering.

Embodiment 2. A method for reducing the frequency, volume, or frequency and volume of blood transfusions in a burn patient in need thereof following a surgical excision of burn wounds comprising administering at least two units of oxygen and carbon dioxide reduced blood to the burn patient during the surgical excision of the burn wounds, the oxygen and carbon dioxide reduced blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the at least two units of oxygen and carbon dioxide reduced blood are administered during the surgical excision of the burn wounds, wherein the average frequency of subsequent blood transfusions after the administering is reduced by at least 10%, the average volume of subsequent blood transfusions after the administering is reduced by at least 10% and the frequency is reduced by at least 10%, or both the average frequency of subsequent blood transfusions after the administering is reduced by at least 10% and the average volume of subsequent blood transfusions after the administering is reduced by at least 10%

Embodiment 3. The method of Embodiment 2, wherein a transfusion-related adverse event does not occur for at least 7 days after the administering.

Embodiment 4. The method of Embodiment 1, wherein the risk of the transfusion-related adverse event occurring in the burn patient is reduced by at least 25% compared to a burn patient administered an identical volume of conventional blood.

Embodiment 5. The method of any one of Embodiments 1 to 4, wherein the burn patient has a total body surface burn area of less than 50%.

Embodiment 6. The method of Embodiment 5, wherein the burn patient has a total body surface burn area of less than 25%.

Embodiment 7. The method of any one of Embodiments 1 to 6, wherein the burn patient is undergoing surgical excision of burn wounds during the administering.

Embodiment 8. The method of Embodiment 7, wherein the burn patient is undergoing surgical grafting of burn wounds during the administering.

Embodiment 9. The method of any one of Embodiments 1 to 6, wherein the burn patient has undergone surgical excision of burn wounds prior to the administering.

Embodiment 10. The method of Embodiment 9, wherein the burn patient has undergone surgical grafting of burn wounds prior to the administering.

Embodiment 11. The method of any one of Embodiments 1 to 10, wherein the burn patient has a burn-related complication selected from the group consisting of excessive histamine release, decreased blood pressure, and excessive bleeding prior to the administering.

Embodiment 12. The method of Embodiment 11, wherein the burn-related complication is improved by at least 50% after the administering.

Embodiment 13. The method of any one of Embodiments 1 to 12, wherein the burn patient has compartment syndrome, multiple organ dysfunction, or both prior to the administering.

Embodiment 14. The method of Embodiment 13, wherein the compartment syndrome, multiple organ dysfunction, or both are stabilized after the administering.

Embodiment 15. The method of any one of Embodiments 1 to 14, wherein the burn patient has a hemoglobin level of less than 9 grams per deciliter (g/dL) prior to the administering.

Embodiment 16. The method of any one of Embodiments 1 to 15, wherein the burn patient has a hemoglobin level of greater than 9 grams per deciliter (g/dL) after the administering.

Embodiment 17. The method of any one of Embodiments 1 to 16, wherein the hemoglobin level of the burn patient is increased by at least 1.0 grams per deciliter (g/dL) after the administering.

Embodiment 18. The method of any one of Embodiments 1 to 17, wherein the administering is over a period of about two hours.

Embodiment 19. The method of any one of Embodiments 1, and 3 to 18, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), haematoma, arterial puncture, delayed bleeding, localized infection or inflammation, brachial artery pseudoaneurysm, deep vein thrombosis (DVT), vasovagal reactions, nerve injury or irritation, arteriovenous fistula, and any combination thereof.

Embodiment 20. The method of any one of Embodiments 1, and 3 to 19, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 21. The method of any one of Embodiments 1, and 3 to 20, wherein the transfusion-related adverse event does not occur for at least 28 days after the administering.

Embodiment 22. The method of any one of Embodiments 1 to 21, wherein the storage period is less than 2 days, less than 7 days, less than 14 days, less than 21 days, less than 28 days, less than 35 days, less than 42 days, or 42 days.

Embodiment 23. The method of any one of Embodiments 1 to 22, wherein the at least two units of oxygen and carbon dioxide reduced, leukoreduced red blood cells further comprise a mixture of citrate-phosphate-dextrose (CPD) and phosphate-adenosine-guanosine-glucose-saline-mannitol (PAGG-SM).

Embodiment 24. The method of any one of Embodiments 1 to 23, wherein the SO₂ is 10% or less.

Embodiment 25. The method of any one of Embodiments 1 to 24, wherein the pCO₂ is 15 mmHg or less.

Embodiment 26. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume for a burn patient in need thereof.

Embodiment 27. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of increasing the blood transfusion interval for a burn patient in need thereof.

Embodiment 28. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume, increasing the blood transfusion interval, or both in a burn patient in need thereof.

Embodiment 29. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of preventing or reducing the risk of a transfusion-related adverse event in a burn patient in need thereof.

Embodiment 30. The use of Embodiment 29, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), haematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 31. The use of Embodiment 29, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 32. The method of Embodiment 1 or Embodiment 2, wherein the blood is selected from the group consisting of whole blood, leukoreduced red blood cells (RBCs), platelet reduced RBCs, leukocyte and platelet reduced RBCs, packed red blood cells, platelet reduced packed red blood cells, leukocyte reduced packed red blood cells, and leukocyte and platelet reduced packed red blood cells.

Embodiment 33. The method of Embodiment 1 or Embodiment 2, wherein said blood comprises red blood cells.

Embodiment 34. The method of Embodiment 1 or Embodiment 2, wherein said blood comprises leukoreduced red blood cells.

### EXAMPLES

### Example 1: Testing of Red Blood Cell Infusion

Fourteen (14) participants receive two separate infusions of approximately 10 mL of autologous radiolabeled red blood cells (RBCs). One of the infusions is with hypoxic RBCs in AS-3 produced using the Hemanext ONE system, as outlined in International Publication Nos. WO 2016/145210 A1 and WO 2016/172645 A1 (incorporated herein by reference) and the other transfusion is with conventional RBCs in AS-3. The hypoxic RBCs infused have an overall higher dual-label 24 hour in vivo recovery (mean: 89.3, SD: 5.81) than the conventional RBCs (mean: 85.8, SD: 6.12). None of the participants experience any adverse events (AE) or serious adverse events (SAE) related to the use of hypoxic RBCs.

### Example 2: Treatment of Burn Patients

Ten burn patients, who require transfusion of red blood cells and who fulfill eligibility criteria are enrolled in a study to test the safety of oxygen and carbon dioxide reduced and leukoreduced red blood cells for transfusion. Patients meeting the following criteria are included in the clinical investigation and undergo the informed consent process:
1. Male or female patients at least 18 years of age.
2. Patients hospitalized with a total body surface area (TBSA %) burn less than 50%.
3. Patients expected to require >2 unit of red blood cells in a single transfusion event.
4. Patients who have the capacity to consent to participate or have a legally authorized representative agreeing to provide consent to participate.

Patients meeting any of the following exclusion criteria are excluded from participation in the clinical investigation:
1. Patients with any positive antibody screening test.
2. Patients for whom consent has not been obtained.
3. Patients with a known hemolytic anemia (congenital or acquired).
4. Patients <18 years old.
5. Patients with a known or suspected pregnancy.
6. Patients with a history of major transfusion reactions.
7. Patients whom the Investigator deems clinical trial participation is not in their best interest.
8. Patients hospitalized with a Total body surface area (TBSA %) burn greater than or equal to 50%.
9. Patients with combined trauma in need of blood transfusions for injuries other than the burn excision.

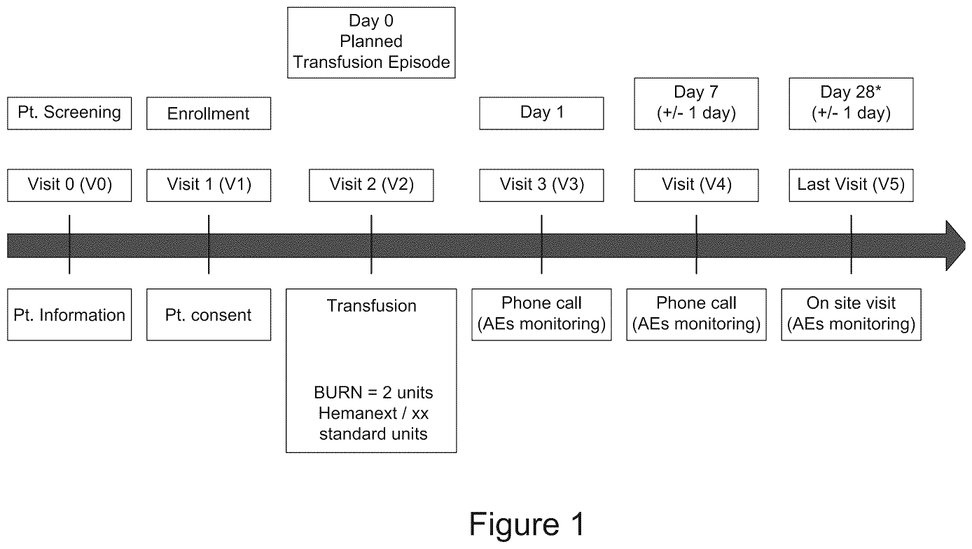

Patients are transfused with 2 units of hypoxic blood (i.e., oxygen reduced blood, or oxygen and carbon dioxide reduced blood) during scheduled transfusion therapy visits. The remainder of the units required for transfusion are drawn from traditionally processed red cells. Adverse events are collected from the Screening visit through either discharge, the subsequent transfusion, or Day 28, whichever comes first. See FIG. 1. Patients are evaluated over five visits. The primary objective of the study is to determine the safety and tolerance of hypoxic RBCs up to 24 hours following the transfusion initiation and overall up to 7 days (+/−1 day) after the transfusion episode (single transfusion course). The secondary objectives are as follows:
1. Assessment of pre- and post-transfusion hemoglobin levels;
2. Assessment of hemoglobin level before the subsequent transfusion, if applicable;
3. Assessment of the occurrence of adverse events (AEs):
   a. Up to 7 days (+/−1 day) post transfusion, in comparison with historical control published in local registries (including but not limited to infection, deep vein thrombosis, acute respiratory distress syndrome, transfusion-related acute lung injury, transfusion associated circulatory overload, anaphylactic shock, acute hemolytic transfusion reaction).
   b. Up to the subsequent transfusion episode or up to 28 days after the initial transfusion, whichever comes first.
   c. From enrollment, up to their subsequent transfusion or 28 days post-transfusion, whichever comes first, through the assessment of patient's diary;
4. Assessment of the vital signs during and up to 15 minutes after the transfusion.
5. Assessment of arterial blood gases pre-operative, intra-operative, and postoperative.

The primary outcome is to determine the number of participants who experience an adverse event (all types/grades) within a time frame. Patients are monitored for adverse events throughout the entirety of their participation in the clinical investigation. Investigators monitor patients directly for adverse events during their transfusion visit. In addition, patients are contacted by phone 24 hours and 7 days (+/−1) after a transfusion visit to record any adverse events experienced since their transfusion visit. All adverse events are recorded.

Patients are monitored for adverse events throughout the entirety of their participation in the clinical investigation. Adverse events are elicited from the patients, the medical record, and an adverse event diary. Enrolled patients undergo standard care for their condition as prescribed by the Investigators. All procedures are performed in compliance with the site's established policies and work instructions. The type and the grade of each adverse event are categorized according to:
1. Association for the Advancement of Blood and Biotherapies (AABB) technical manual, 20th edition (2020);
2. Delaney et al., “Biomedical Excellence for Safer Transfusion (BEST) Collaborative. Transfusion reactions: prevention, diagnosis, and treatment,” Lancet 388:2825-2836 (2016);
3. Local AEs database;
4. ISO 14155-2020 (Clinical investigation of medical devices for human subjects—Good Clinical Practice) definitions.

Patients are also monitored for secondary outcomes related to AE occurrence during their transfusion visit and then patients are contacted by phone 24 hours and 7 days (+/−1) after their transfusion visit to record any adverse events they experienced since their transfusion visit. The patient's chart is checked for any AE occurrence after their following transfusion visit or 28 days after their initial transfusion, whichever comes first. All adverse events are recorded.

The secondary outcomes measured include, but are not limited to, the following:
1. Evolution of the hemoglobin level before and after the transfusion;
2. Calculation of the hemoglobin increment after transfusion corrected for patient blood volume and hemoglobin dose;
3. Comparison of the hemoglobin level before the index transfusion to that prior to the subsequent transfusion;
4. Comparison of the frequency of all AEs, including transfusion related AEs up to 7 days (+/−1) post-transfusion compared to historical data in local patient registries (for example, The National Burn Registry);
5. Evaluation of AEs from enrollment, up to prior to the subsequent transfusion or up to 28 days after the initial transfusion, whichever comes first;
6. Evaluation of the frequency of all AEs, from enrollment, up to their subsequent transfusion or 28 days post-transfusion, whichever comes first, through the assessment of patient's diary, when applicable;
7. Evaluation of patient's vital signs over the course of the transfusion and up to 15 minutes post-transfusion.

Patient information is collected from enrollment and throughout the end of the study visit as outlined in Table 1.

TABLE 1
Burn Patient Screening, Transfusion, and Follow-up Guidelines

| | Investigation Periods | | | | | | |
|---|---|---|---|---|---|---|---|
| | Screening & | | | | | | |
| | Enrollment | Pre- | | Post- | Follow Up | Follow-Up | Follow-Up |
| | Visit | Transfusion | Transfusion | Transfusion | 1 | 2 | 2 |
| | Visit | | | | | | |
| | 1 | 2 | 2 | 2 | 3 | 4 | 5 |
| | Time (hour, day, week) | | | | | | |
| | | | | | | | Day of |
| | | | | | | | subsequent |
| | | | | | | | transfusion |
| | | | | | Day 2 (≤24 | Day 7 (+/−1 | or D 28, |
| | Day 0 | Day 1 | Day 1 | Day 1 | h) | day) | whicheve [symbol] |
| In-/Exclusion | x | | | | | | |
| Criteria | | | | | | | |
| TBSA | x | | | | | | |
| Patient Information | | | | | | | |
| Patient information | x | | | | | | |
| and consent (ICF) | | | | | | | |
| Patient legally | x (if | | | | | | |
| authorized | applicable) | | | | | | |
| representative | | | | | | | |
| information and | | | | | | | |
| consent (ICF) | | | | | | | |
| Demographics | x | | | | | | |
| Medical History | x | | | | | | |
| Concomitant | x | x | x | x | x | | x |
| Medications | | | | | | | |
| Physical | x | | | | | | x |
| Examination | | | | | | | |
| Pregnancy Test | x | | | | | | |
| Height | x | | | | | | |
| Weight | x | | | | | | |
| RBC Unit Weight | | x | | | | | |
| Patient | | x | | x | | | x |
| Hemoglobin | | | | | | | |
| Blood Pressure | x | x | x | x | | | |
| Heart Rate | x | x | x | x | | | |
| Body Temperature | x | x | x | x | | | |
| Respiratory Rate | x | x | x | x | | | |
| Oxygen Saturation | x | x | x | x | | | |
| Transfusion | | | x | | | | |
| Adverse | x | x | x | x | x | x | x |
| Events, | | | | | | | |
| Adverse | | | | | | | |
| Review Device | | x | x | x | | | x |
| (blood bag) | | | | | | | |

[symbol]

indicates data missing or illegible when filed

During the first follow-up visit 24 hours post-transfusion infection patients are evaluated for infection occurrence, adverse event occurrence, concomitant medications, and device deficiencies. The second follow-up visit occurs over the phone 7 days (+/−1) post-transfusion. Patients are evaluated for the occurrence of infection, adverse event(s), concomitant medications, device deficiencies, and patient diary. The final follow-up occurs at the patient's subsequent transfusion visit or 28 days after transfusion, whichever comes first. Patients are evaluated for the occurrence of adverse event(s) and concomitant medications, pre-transfusion hemoglobin, and a physical examination is performed.

### Example 3: Treatment of Perioperative Bleeding (Burn Patients)

Four patients aged >18 years who require >2 units of RBCs due to perioperative bleeding resulting from burns receive one 2-hour transfusion episode of two units of hypoxic RBCs produced using the CPD/PAGGSM, leukoreduced (LR), O₂/CO₂ reduced system instead of conventionally stored RBCs. Patient characteristics and pre- and post-transfusion hemoglobin levels are provided in Table 2 (all plus-minus values are means±standard deviation (SD)).

TABLE 2
Characteristics of Patients with Perioperative
Bleeding (Burn Patients)

| Sex - Number | |
|---|---|
| Male | 3 |
| Female | 1 |
| Age, years | 44 ± 13.52 |
| Body Mass Index (BMI), kg/m² | 33.35 ± 10.83 |
| Units of Hypoxic Red Blood Cells (RBCs) | 2 |
| Pre-Transfusion Hemoglobin (Hb), g/dL | 10.38 ± 1.25 |
| Post-Transfusion Hb (1 hour), g/dL | 11.18 ± 1.43 |

The number of adverse events (AEs) up to 24 hours following transfusion initiation and up to 7 days (±1 day) post-transfusion are compared with historical controls (including but not limited to infection, deep vein thrombosis, acute respiratory distress syndrome, transfusion-related acute lung injury, transfusion associated circulatory overload, anaphylactic shock, and acute hemolytic transfusion reaction). All patients receive hypoxic RBCs without complication. No adverse events occur in the patients that are deemed related to the hypoxic RBCs, and no severe adverse events are observed. No reports of deep vein thrombosis, acute respiratory distress syndrome, transfusion-related acute lung injury, transfusion-associated circulatory overload, anaphylactic shock, or acute hemolytic transfusion reaction are observed. Hemoglobin levels increase by about 8% one hour after administration of hypoxic RBCs. These results demonstrate that transfusion of hypoxic RBCs produced by the CPD/PAGGSM, LR, O₂/CO₂ reduced system are safe and well-tolerated in patients with perioperative bleeding resulting from burns. Further, hypoxically stored RBCs function appropriately upon transfusion to burn patients with perioperative bleeding, as indicated by the changes in Hb levels post-transfusion.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure.

Therefore, it is intended that the present disclosure not be limited to the particular embodiments disclosed as the best mode contemplated for carrying out the present disclosure, but that the present disclosure will include all embodiments falling within the scope and spirit of the appended claims.

1. A method for reducing the risk or preventing the occurrence of a transfusion-related adverse event in a burn patient in need thereof comprising administering at least two units of oxygen and carbon dioxide reduced, leukoreduced red blood cells to the burn patient, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the at least two units of oxygen and carbon dioxide reduced, leukoreduced red blood cells are administered to the burn patient in a single transfusion event, and wherein the transfusion-related adverse event does not occur for at least 7 days after the administering.

2. A method for reducing the frequency, volume, or frequency and volume of blood transfusions in a burn patient in need thereof following a surgical excision of burn wounds comprising administering at least two units of oxygen and carbon dioxide reduced, leukoreduced red blood cells to the burn patient during the surgical excision of the burn wounds, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the at least two units of oxygen and carbon dioxide reduced, leukoreduced red blood cells are administered during the surgical excision of the burn wounds, wherein the average frequency of subsequent blood transfusions after the administering is reduced by at least 10%, the average volume of subsequent blood transfusions after the administering is reduced by at least 10% and the frequency is reduced by at least 10%, or both the average frequency of subsequent blood transfusions after the administering is reduced by at least 10% and the average volume of subsequent blood transfusions after the administering is reduced by at least 10%.

3. The method of claim 2, wherein a transfusion-related adverse event in the burn patient does not occur for at least 7 days after the administering.

4. The method of claim 1, wherein the risk of the transfusion-related adverse event occurring in the burn patient is reduced by at least 25% compared to a burn patient administered an identical volume of conventional red blood cells.

5. The method of claim 1, wherein the burn patient has a total body surface burn area of less than 50%.

6. The method of claim 5, wherein the burn patient has a total body surface burn area of less than 25%.

7. The method of claim 1, wherein
a. the burn patient is undergoing surgical excision of burn wounds during the administering; or
b. the burn patient is undergoing both surgical excision and surgical grafting of burn wounds during the administering.

8. (canceled)

9. The method of claim 1, wherein
a. the burn patient has undergone surgical excision of burn wounds prior to the administering; or
b. the burn patient has undergone both surgical excision and surgical grafting of burn wounds prior to the administering.

10. (canceled)

11. The method of claim 1, wherein the burn patient has a burn-related complication selected from the group consisting of excessive histamine release, decreased blood pressure, and excessive bleeding prior to the administering.

12. The method of claim 11, wherein the burn-related complication is improved by at least 50% after the administering.

13. The method of claim 1, wherein the burn patient has compartment syndrome, multiple organ dysfunction, or both prior to the administering.

14. The method of claim 13, wherein the compartment syndrome, multiple organ dysfunction, or both are stabilized after the administering.

15. The method of claim 1, wherein the burn patient has a hemoglobin level of less than 9 grams per deciliter (g/dL) prior to the administering.

16. The method of claim 15, wherein
a. the hemoglobin level is greater than 9 g/dL after the administering; or
b. the hemoglobin level is increased by at least 1.0 g/dL after the administering.

17. (canceled)

18. The method of claim 1, wherein the administering is over a period of about two hours.

19. The method of claim 1, wherein
a. the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), haematoma, arterial puncture, delayed bleeding, localized infection or inflammation, brachial artery pseudoaneurysm, deep vein thrombosis (DVT), vasovagal reactions, nerve injury or irritation, arteriovenous fistula, and any combination thereof; or
b. the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

20. (canceled)

21. The method of claim 1, wherein the transfusion-related adverse event does not occur for at least 28 days after the administering.

22. The method of claim 1, wherein the storage period is less than 42 days.

23. The method of claim 1, wherein the at least two units of oxygen and carbon dioxide reduced, leukoreduced red blood cells further comprise a mixture of citrate-phosphate-dextrose (CPD) and phosphate-adenosine-guanosine-glucose-saline-mannitol (PAGG-SM).

24. The method of claim 1, wherein
a. the SO₂ is 10% or less,
b. the pCO₂ is 15 mmHg or less; or
c. both a and b.

25. (canceled)
